(19) United States
(12) Patent Application Publication
(10) Pub. No.: US 2026/0344979 A1
(43) Pub. Date: Nov. 5, 2026

(54) ENDOSCOPE VALVES

(71) Applicant: Boston Scientific Medical Device Limited, Galway (IE)

(72) Inventor: Bosong Xu, Suzhou (CN)

(73) Assignee: Boston Scientific Medical Device Limited, Galway (IE)

(21) Appl. No.: 19/664,523

(22) Filed: Apr. 30, 2026

(30) Foreign Application Priority Data
May 2, 2025 (CN) 202510575145.2

(51) Int. Cl. A61B 1/00

(52) U.S. Cl. CPC A61B 1/00068

(57) ABSTRACT

The disclosure relates to medical devices and medical device valves. An example medical device may include a handle, an elongate shaft extending from the handle and configured to be inserted into a body cavity of a patient, a suction valve configured to be removably positioned in a first valve body at the handle, and a gas/water valve configured to be positioned in a second valve body at the handle. The second valve body may be configured to block removal of the gas/water valve from the second valve body. The medical device may be a single use medical device. The medical device may be an endoscope.

### CROSS-REFERENCE TO RELATED APPLICATIONS

This application claims the benefit of Chinese Patent Application Serial No. 202510575145.2 filed on May 2, 2025, the disclosure of which is incorporated herein by reference.

### TECHNICAL FIELD

This disclosure relates generally to valve assemblies and methods, and particularly to endoscopes valves and methods of thereof.

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems.

In an example, a valve assembly for use in a valve well of a medical device may be provided. The valve assembly may include a housing, an interface member, a valve stem configured to adjust between a first position and a second position in response to actuation of the interface member relative to the housing, a first seal coupled with the housing and a second seal coupled with the valve stem, wherein the valve stem may be configured to engage the first seal when the valve stem is in the first position and the second seal is configured to engage the housing when the valve stem is in the second position.

Alternatively or additionally to any of the examples above, the second seal may be configured to be proximal of the first seal when the valve stem is in the first position and when the valve stem is in the second position.

Alternatively or additionally to any of the examples above, the housing may comprise a radial opening, the radial opening may be distal of a proximal opening through the housing and is in fluid communication with the proximal opening through the housing when the valve stem is in the first position, and the radial opening may be proximal of a distal opening through the housing and is in fluid communication with the distal opening through the housing when the valve stem is in the second position.

Alternatively or additionally to any of the examples above, the first seal may extend around a circumference of the distal opening through the housing.

Alternatively or additionally to any of the examples above, the interface member may be located in the proximal opening.

Alternatively or additionally to any of the examples above, the valve stem may have a distal portion, a proximal portion, and an intermediate portion between the proximal portion and the distal portion, and the distal portion may have a first cross-sectional area configured to create a seal engagement with the first seal when the valve stem is in the first position and the intermediate portion has a second cross-sectional area configured to extend through the first seal and allow fluid to flow between the valve stem and the first seal when the valve stem is in the second position.

Alternatively or additionally to any of the examples above, the proximal portion may have a third cross-sectional area and the second cross-sectional area of the intermediate portion may be less than the first cross-sectional area and the third cross-sectional area.

Alternatively or additionally to any of the examples above, the intermediate portion may have a constant cross-sectional area between the distal portion and the proximal portion.

Alternatively or additionally to any of the examples above, the housing may include a first portion configured to extend into the valve well and a second portion configured to receive the interface member.

Alternatively or additionally to any of the examples above, the first portion may have a first inner diameter and the second portion has a second inner diameter that is greater than the first inner diameter.

Alternatively or additionally to any of the examples above, the valve assembly may include a biasing member extending between the housing and the interface member, wherein the biasing member may be configured to bias the interface member to a first configuration with the valve stem in the first position.

In another example, a valve body having one or more fluid inlet ports and one or more fluid outlet ports, an interface member extending into the valve body, a valve stem connected to the interface member and configured to translate within the valve body between a first position and a second position in response to actuation of the interface member, the valve stem comprising an elongate body and a central lumen extending through the elongate body between a first opening in the elongate body and a second opening in the elongate body, and one or more seals extending around the valve stem and configured to block fluid flow between an outer surface of the valve stem and an inner surface of the valve body, wherein the interface member may be configured to be inserted into the valve body and the valve body is configured to block removal of the interface member from the valve body.

Alternatively or additionally to any of the examples above, the valve body may include a proximal opening through which the interface member is inserted.

Alternatively or additionally to any of the examples above, the valve body may include a first engagement feature and the interface member comprises a second engagement feature configured to engage the first engagement feature when the valve stem is in the first position to block removal of the interface member from the valve body.

Alternatively or additionally to any of the examples above, the second engagement feature may comprise a beveled outer surface that tapers inward in a distal direction and a proximal shoulder configured to engage the first engagement feature.

Alternatively or additionally to any of the examples above, first engagement feature may extend around an inner circumference of the valve body.

Alternatively or additionally to any of the examples above, the valve body may be configured to receive the interface member and receive the valve stem.

In another example, a single-use medical device may include a handle, an elongate shaft extending from the handle and configured to be inserted into a body cavity of a patient, a suction valve configured to be removably positioned in a first valve body at the handle, and a gas/water valve configured to be positioned in a second valve body at the handle, wherein the second valve body is configured to block removal of the gas/water valve from the second valve body.

Alternatively or additionally to any of the examples above, the suction valve may include a housing inserted into the first valve body, a valve stem configured to adjust between a first position and a second position relative to the first valve body, a first seal coupled with the housing, and a second seal coupled with the valve stem, wherein the valve stem may be configured to engage the first seal when the valve stem is in the first position and the second seal is configured to engage the housing when the valve stem is in the second position.

Alternatively or additionally to any of the examples above, the gas/water valve may include an interface member extending into the second valve body and a valve stem connected to the interface member and configured to translate within the second valve body between a first position and a second position in response to actuation of the interface member, wherein the second valve body may include a first engagement feature and the interface member may include a second engagement feature configured to engage the first engagement feature when the valve stem is in the first position to block removal of the gas/water valve from the second valve body.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various configurations and together with the description serve to explain the principles of the present disclosure.

FIG. 1 depicts a schematic view of components of an illustrative endoscope;

FIG. 2 depicts a schematic view of components of an illustrative endoscope system;

FIG. 3 depicts a schematic side view of an illustrative valve for an endoscope;

FIG. 4 depicts a schematic cross-section view of the valve depicted in FIG. 3;

FIG. 5 depicts a schematic side view of an illustrative valve stem and interface of a valve for an endoscope;

FIG. 6 depicts a schematic side view of the valve stem and interface member depicted in FIG. 5 and rotated 90 degrees;

FIG. 7 depicts a schematic side view of an illustrative valve for an endoscope;

FIG. 8 depicts a schematic bottom perspective view of the valve depicted in FIG. 7;

FIG. 9 depicts a schematic top perspective view of the valve depicted in FIG. 7;

FIG. 10 depicts a schematic bottom perspective view of components of the valve depicted in FIG. 7; and

FIG. 11 depicts a schematic top perspective view of components of the valve depicted in FIG. 7.

While the disclosure is amenable to various modifications and alternative forms, specifics thereof have been shown by way of example in the drawings and will be described in detail. It should be understood, however, that the intention is not to limit the invention to the particular configurations described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed configuration(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “a configuration”, “some configurations”, “other configurations”, etc., indicate that the configuration(s) described may include a particular feature, structure, or characteristic, but every configuration may not necessarily include the particular feature, structure, or characteristic. Moreover, such phrases are not necessarily referring to the same configuration. Further, when a particular feature, structure, or characteristic is described in connection with a configuration, the particular feature, structure, or characteristic may be effected in connection with other configurations, whether or not explicitly described, unless clearly stated to the contrary. That is, the various individual elements described below, even if not explicitly shown in a particular combination, are nevertheless contemplated as being combinable or arrangeable with each other to form other additional configurations or to complement and/or enrich the described configuration(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. The numerical nomenclature is not intended to be limiting and is illustrative only. In some configurations, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element.

The detailed description is intended to illustrate but not limit the disclosure. The various elements described may be arranged in various combinations and configurations without departing from the scope of the disclosure. The detailed description illustrates example configurations of the disclosure.

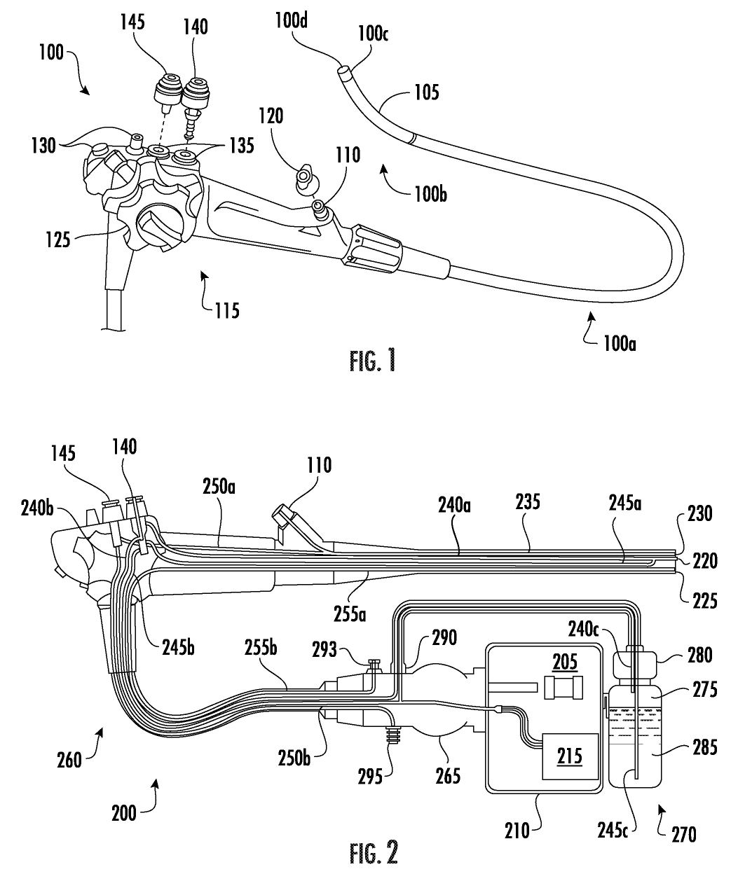

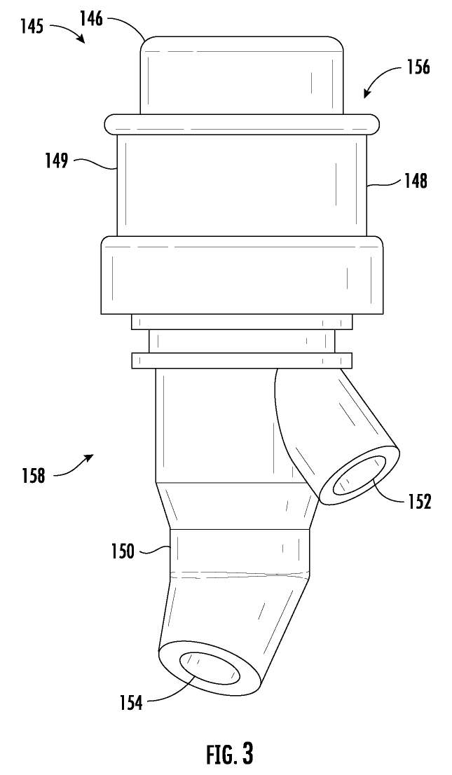

With reference to FIG. 1, an illustrative endoscope 100 is schematically depicted and FIG. 2 schematically depicts an illustrative endoscope system 200. Endoscope 100 may include an elongated tube or shaft 100a that is configured to be inserted into a subject (e.g., a patient).

Although endoscopes (and combination devices that perform functions of endoscopes) are particularly referenced herein, the disclosure also encompasses other types of devices, such as duodenoscopes, choledochosopes, bronchoscopes, gastroscopes, endoscopic ultrasound (“EUS”) scopes, colonoscopes, ureteroscopes, bronchoscopes, laparoscopes, cystoscopes, aspiration scopes, sheaths, catheters, or similar devices. A reference to a endoscope herein may encompass any of the above medical devices.

A light source 205 of endoscope system 200 may feed illumination light to a distal portion 100b of endoscope 100. The distal portion 100b of endoscope 100 may house an imager (e.g., CCD or CMOS imager) (not shown). Light source 205 (e.g., lamp) may be located in a video processing unit 210 that processes signals input from the imager and outputs processed video signals to a video monitor (not shown) for viewing. The video processing unit 210 may also serve as a component of a gas/water feed circuit by housing a pressurizing pump (e.g., an air pump 215), such as an air feed pump, in video processing unit 210.

Endoscope shaft 100a may include a distal tip 100c (e.g., a distal tip unit adapted to be inserted into a body cavity of a patient) provided at distal portion 100b of shaft 100a and a flexible bending portion 105 proximal to distal tip 100c. Flexible bending portion 105 may include an articulation joint (not shown) to assist with steering distal tip 100c. On an end face 100d of distal tip 100c of endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along shaft 100a for passing tools to the treatment area, may also be included on the face 100d of the distal tip 100c. Working channel 235 may extend along shaft 100a to a proximal channel opening 110 positioned distal to an operating handle 115 (e.g., a proximal handle) of endoscope 100. A biopsy valve 120 may be utilized to seal channel opening 110 against unwanted fluid egress.

Operating handle 115 may be provided with knobs 125 for providing remote 4-way steering of the distal tip via wires connected to the articulation joint in the flexible bending portion 105 (e.g., one knob controls up-down steering and another knob controls left-right steering). One or more (e.g., one, two, a plurality, etc.) of video switches 130 for remotely operating the video processing unit 210 may be arranged on a proximal end side of the handle 115.

Handle 115 may be provided with dual valve locations 135. One of the valve locations 135 may have or receive a gas/water valve 140 (e.g., an air/water valve, etc.) for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220, as depicted in FIG. 2.

The other valve location 135 may have or receive a suction valve 145 for operating a suction operation. A suction supply line 250a may run distally from the suction valve 145 along shaft 100a to a junction point in fluid communication with the working channel 235 of the endoscope 100.

The operating handle 115 may be electrically and fluidly connected to video processing unit 210, via a flexible umbilical 260 and connector portion 265 extending therebetween. The flexible umbilical 260 has a gas (e.g., air or CO2) feed line 240b, a lens wash feed line 245b, a suction feed line 250b, an irrigation feed line 255b, a light guide (not shown), and an electrical signal cable (not shown). The connector portion 265 when plugged into the video processing unit 210 connects light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects air pump 215 to the gas feed line 240b in the umbilical 260.

A water container or reservoir 270 (e.g., a water bottle and/or other suitable reservoir or container) may be fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, may pass through the top 280 of the reservoir 270 to the same detachable connection 290 as the gas supply tubing 240c on the connector portion 265. In other configurations, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some configurations, irrigation water is supplied via a pump (e.g., peristaltic pump) from a water source independent (not shown) from the water reservoir 270. In other configurations, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve 140 in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

Referring to FIG. 2, an illustrative operation of an endoscope system 200, including an endoscope such as the endoscope 100 depicted in FIG. 1, is explained. Air from the air pump 215 in the video processing unit 210 may flow through the connector portion 265 and branch to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user’s finger on the valve, air is allowed to flow out of the valve 140 to atmosphere. In a first position, the user’s finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve 140, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some configurations, a filter (not shown) may be placed in the path of the gas supply tubing 240c to filter-out undesired contaminants or particulates from passing into the water reservoir 270. In some configurations, outflow check valves or other one-way valve configurations (not shown) may be placed in the path of the lens wash supply tubing to help prevent water from back-flowing into the reservoir 270 after the water has passed the valve.

A relatively higher flow rate compared to lens wash is typically required for irrigation water, since a primary use is to clear the treatment area in the patient of debris that obstructs the user’s field of view. Irrigation is typically achieved with the use of a pump (e.g., peristaltic pump), as described. In configurations with an independent water source for irrigation, tubing placed in the bottom of a water source may be passed through the top of the water source and threaded through the head on the upstream side of the pump. Tubing on the downstream side of the pump is connected to the irrigation feed line 255b in the umbilical 260 and the irrigation supply line 255a of endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical 260, and down the irrigation supply line in the shaft 100a of the endoscope 100 to the distal tip 100c. In order to equalize the pressure in the water source as water is pumped out of the irrigation supply tubing, an air vent (not shown) may be included in the top 280 of water reservoir 270. The vent allows atmospheric air into the water source preventing negative pressure build-up in the water source, which could create a vacuum that suctions undesired matter from the patient back through the endoscope toward the water source. In some configurations, outflow check valves or other one-way valve configurations (not shown), similar to the lens wash tubing 245c, may be placed in the path of the irrigation supply tubing to help prevent back-flow into the reservoir after water has passed the valve.

The suction valve 145 may be configured to allow or prevent suction and/or a suction effect in the working channel 235. When the suction valve 145 is in a valve closed position (e.g., a first configuration), a suction fluid flow through the working channel 235 may be blocked by the suction valve 145. When suction is desired in the working channel 235, an operator or user may actuate the suction valve 145 (e.g., by depressing a button on the valve and/or actuating the suction valve 145 in one or more other suitable manners) in order to bring the suction valve 145 to a valve open position (e.g., a second configuration). When the suction valve 145 is in the valve opened position, a flow channel inside the suction valve 145 may connect the working channel 235 to the suction device coupled to suction connection 295 and the suction device may create a negative pressure that draws fluid into and out of the working channel 235 through an outlet provided in the suction valve. When the operator or user releases the suction valve 145, the valve 145 may return to its valve closed position and reduce or block a suction fluid flow from the working channel 235.

In some cases, suction valves 145 may rely on a path of least resistance to direct a suction fluid flow through the endoscope system 200. In some cases, when a suction pump is turned on for a procedure, the pump remains on for an entirety of the procedure and continually pulls air from the flexible umbilical 260, which in turn draws fluid from the line side of the endoscope 100 that runs up the umbilical 260 and connects to a port at the suction valve 145. When the suction valve 145 is in a first position and/or configuration (e.g., a closed position) the suction force or negative pressure from the suction pump is blocked from the working channel 235 and may pull fluid from atmosphere through the suction valve 145. When the suction valve 145 is actuated to a second position and/or configuration (e.g., an opened position) (e.g., when the button or cap associated with the suction valve 145 is depressed and/or actuated in one or more other suitable manners), the opening from atmosphere through the suction valve 145 to the suction pump may be effectively closed or blocked by the suction valve 145 and a fluid path between working channel 235 and the suction pump through the suction valve 145 may be opened. Thus, fluid moving to the suction pump may follow a path of least resistance, where the path may change depending on whether the suction valve 145 is in a first position (e.g., a closed position) or a second position (e.g., an opened position).

Endoscopes include functionality to deliver fluids (including air and water) and suction to a site of a procedure. Tubing for delivering fluids and/or suction extends from a handle of the endoscope, through a sheath of the endoscope, and to a distal tip of the endoscope. During a procedure, body fluids, tissues, or other material can build up in the tubing and, in some cases, lead to clogging of the tubing. After use in a procedure, single-use endoscopes may be discarded or recycled and reusable endoscopes may reprocessed for subsequent use.

FIG. 3 depicts a schematic side view of an illustrative suction valve 145 configured for use in the endoscope 100. In some examples, the suction valve 145 may be used in a single-use configuration of the endoscope 100. In some examples, the suction valve 145 may include a valve stem (not shown in FIG. 3) configured to be removed from a valve well 158 of the suction valve 145 to facilitate removing or otherwise addressing clogs or debris in or through the suction valve 145.

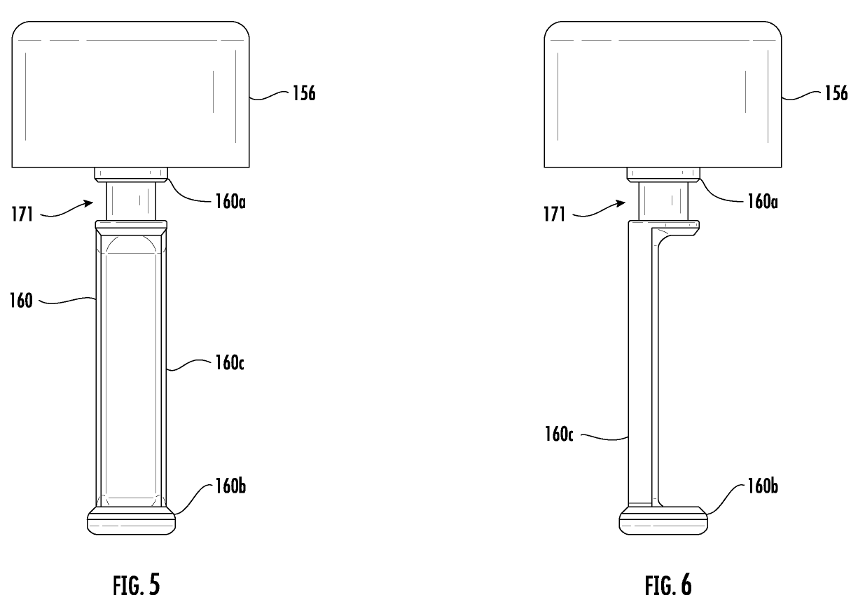

The suction valve 145 depicted in FIG. 3 may include an interface member 146 (e.g., a cap, button, etc.), a housing 148 that may include a collar 149, and a body 150 of or defining the valve well 158, where the body 150 may define a suction port 152 and a working channel port 154. The collar 149 and/or the interface member 146 may at least partially define an opening 156 (e.g., a proximal opening of the valve well 158, as depicted for example in FIG. 6) for fluid from atmosphere (e.g., air) to flow through the suction valve 145. In some examples, the interface member 146 may be located in and/or extend through the opening 156. Although certain components of the suction valve 145 are depicted and described, other suitable components of the suction valve 145 are contemplated and/or certain depicted and described components of the suction valve 145 may be combined to form a single component and/or separated to form multiple components.

The housing 148 and the body 150 may form, be components of, and/or define the valve well 158. Alternatively or additionally, the housing 148 may be coupled with the valve well 158. Although the housing 148 and the body 150 are depicted as separate components, the housing 148 and the body 150 may be formed from a single component, as desired. Other suitable configurations of the housing 148, the body 150, and the valve well 158 are contemplated.

In operation, the suction valve 145 depicted in FIG. 3 (and a valve stem thereof) may have a first position or configuration (e.g., a relaxed or closed position) and a second position or configuration (e.g., an actuated or opened position). When the suction valve 145 is in the first position, the suction valve 145 may be configured to create a fluid path between the opening 156 leading to atmosphere and the suction port 152. When the suction valve 145 is in the second position, the suction valve 145 may be configured to create a fluid path between the working channel port 154 and the suction port 152.

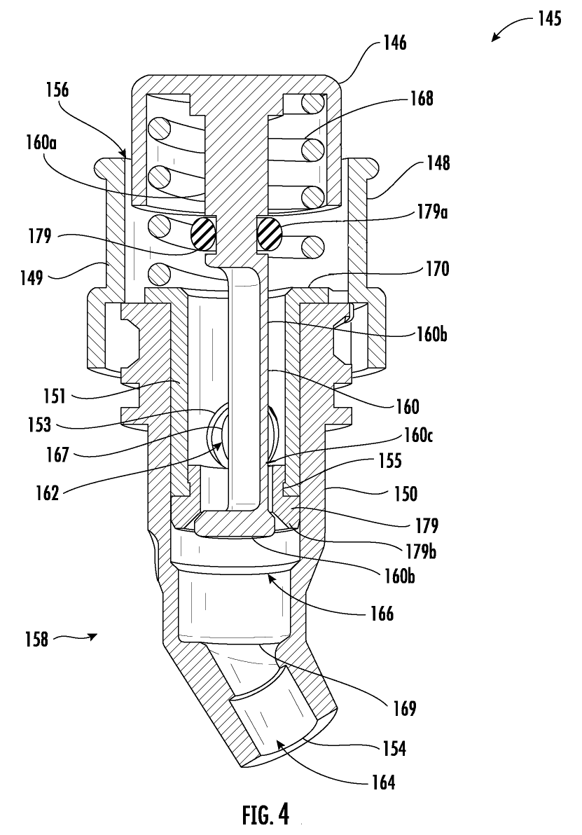

FIG. 4 depicts a schematic cross-section view of the suction valve 145 depicted in FIG. 3, where the suction valve 145 is in a first position (e.g., a relaxed or closed position). As depicted in FIG. 4, the housing 148 may be coupled to the body 150. The housing 148 may be directly or indirectly coupled to the main body 150 in any suitable manner including, but not limited to, via adhesives, a threaded connection, a luer lock connection, a snap connection, a ball-detent connector, a friction fit, an interference fit, and/or additional or alternative coupling techniques. In some examples, the housing 148 may be coupled to the body 150 via an interference fit and/or an adhesive connection. In some examples, the housing 148 may include an insert portion 151 that may be inserted into the valve well 158 and coupled with the body 150. Although other configurations are contemplated, the insert portion 151 may have a diameter that is less than a diameter of the collar 149. Other suitable configurations of coupling the housing 148 with the valve well 158 are contemplated.

The body 150 may define a first lumen 162 (e.g., a suction lumen) to or extending from the suction port 152 (not shown in FIG. 4), define a second lumen 164 (e.g., a working channel lumen) to or extending from the working channel port 154, and a third lumen 166 (e.g., a body lumen) configured to receive the valve stem 160 and in fluid communication with the first lumen 162 and the second lumen 164. In some examples, the third lumen 166 may be configured to receive fluid from atmosphere that travels through the opening 156 to atmosphere and into the third lumen 166 when the suction valve 145 is in the first position. In some examples, the valve well 158 may include a first opening 167 (e.g., a radial opening) leading to the suction port 152 through the first lumen 162, along with a second opening 169 (e.g., an opening at the joint) leading to the working channel port 154 through the second lumen 164. In some examples, the insert portion 151 may include a side or radial opening 153 configured to be aligned with the first opening 167 when the insert portion 151 of the housing 148 is positioned within the body 150 of the valve well 158. In some examples, the side or radial opening 153 may be distal of the opening 156 leading to atmosphere. Additionally or alternatively, the valve well 158 may include one or more other suitable openings and/or lumens and/or configurations of openings and/or lumens.

The body 150 of the valve well 158 depicted in FIG. 4 may be configured to receive a valve stem 160 (e.g., a valve core) of the suction valve 145. In some examples, the valve stem 160 may be in communication with the interface member 146. In some examples, the valve well 158 may be configured to facilitate translation (e.g., longitudinal translation and/or rotational translation) of the valve stem 160 in the body 150 and/or the housing 148 in response to forces acting on the valve stem 160 via the interface member 146. In some examples, the valve well 158 may be in fluid communication with and/or define the suction port 152, the first lumen 162, the working channel port 154, the second lumen 164, and the third lumen 166, where the translation of the valve stem 160 within the valve well 158 may adjust flow paths through the suction valve 145 (e.g., adjust flow paths through the suction port 152, the first lumen 162, the working channel port 154, the second lumen 164, and the third lumen 166).

The suction valve 145 may further include one or more biasing members 168. The biasing members 168 may be any suitable type of biasing member configured to bias the interface 146 to the first position (e.g., as depicted in FIG. 4), allow the interface member 146 to adjust to a second position in response to a force applied to the interface member 146, and return the interface member 146 to the first position when the force applied to the interface member 146 is entirely or at least partially removed from the interface member 146. In one example, the biasing member 168 may be a spring, as depicted in FIG. 4, but other suitable biasing members are contemplated.

When the suction valve 145 includes the interface member 146 and the housing 148, the biasing member 168 may extend between the interface member 146 and the housing 148 (e.g., between the interface member 146 and the insert portion 151 of the housing 148, as depicted in FIG. 4) and/or the body 150 of the valve well 158 to facilitate the interface member 146 adjusting relative to the body 150 and/or the housing 148 (e.g., the collar 149). In some examples, the biasing member 168 may extend between the interface member 146 and a shelf or shoulder 170 of the insert portion 151 of the housing 148. Alternatively or additionally, the biasing member 168 may extend between the interface member 146 and one or more other suitable components of the suction valve 145 to facilitate the interface member 146 adjusting, and thus the valve stem 160 adjusting, relative to the body 150.

The valve stem 160 may couple to the interface member 146 in any suitable manner. In some cases, a first portion 160a(e.g., a proximal portion) of the valve stem 160 extending proximally of a third portion 160c may be coupled to the interface member 146 via one or more suitable coupling mechanisms. For example, when the valve stem 160 is in communication with the interface member 146, the valve stem 160 may be releasably coupled with the interface member 146, monolithically formed with the interface member 146 with the same material(s) forming the interface member 146 (e.g., formed with a single material), fixedly coupled with the interface member 146, and/or may have one or more other suitable configurations with respect to the interface member 146 such that actuation of the interface member 146 may cause actuation or movement of the valve stem 160. Example suitable coupling mechanisms for coupling the valve stem 160 with the interface member 146 include, but are not limited to, adhesives, a threaded connection, a luer lock connection, a snap connection, a ball-detent connector, a friction fit, a forming procedure, a molding technique, and/or additional or alternative coupling mechanisms. In one example, the valve stem 160 may be monolithically formed with the interface member 146 using a single material. Other configurations of the valve stem 160 relative to the interface member 146 are contemplated.

The valve stem 160 may have any suitable configuration configured to adjust positions within the valve well 158, adjust flow paths in communication with the suction port 152, and couple to the interface member 146. For example, the valve stem 160 may be solid, may have one or more openings therethrough and/or therein, may have one or more lumens, and/or may have one or more suitable configurations. In some examples, the valve stem 160 may be solid without any lumens or openings. In one example, the valve stem 160 may be solid with the first portion 160a, a second portion 160b (e.g., a distal portion), and the third portion 160c (e.g., an intermediate portion) extending between the first portion 160a and the second portion 160b, as depicted for example in FIG. 4. In some examples, the third portion 160c may be elongated and have at least one width or diameter dimension that is less than a similar dimension of one or both of the first portion 160a and the third portion 160c. For example, the third portion 160c may define an open channel. In one example, the first portion 160a, the second portion 160b, and the third portion 160c of the valve stem 160 may form an elongate C-shape.

The first portion 160a, the second portion 160b, and the third portion 160c may have any suitable configuration configured or otherwise designed to facilitate adjusting flow paths within the suction valve 145 based on a position of the valve stem 160 relative to the valve well body 150. In one example configuration, the third portion 160c may be configured to cross the first opening 167 from the first portion 160a proximal of the first opening 167 to the second portion 160b distal of the first opening 167, as depicted for example in FIG. 4, when the valve stem 160 is in the first configuration and/or in the second configuration.

The suction valve 145 may include one or more seals 179 configured to interact with (e.g., contact) an inner wall or walls of the valve well 158 and/or to interact with the valve stem 160 to create a barrier to fluid flow. Example suitable configurations of seals 179 include, but are not limited to, valves, annular seals, o-rings, seal pads, resilient material, X-shaped cross-sectional seals, wiper seals, disc shaped seals, pre-configured seals applied to the circumferentially extended recesses, and/or other suitable seal configurations.

Although other suitable numbers of seals 179 may be utilized, the suction valve 145 may include a first seal 179a and a second seal 179b. In some examples, the first seal 179a of the suction valve 145 may be configured to translate with the valve stem 160 and the second seal 179b may be configured to remain stationary relative to the body 150 of the valve well 158 as the valve stem 160 translates. In one example, as depicted in FIG. 4, the first seal 179a may be an o-ring, coupled to the first portion 160a of the valve stem 160, configured to be proximal of the shelf or shoulder 170 when the suction valve 145 is in the first position to allow a flow between atmosphere (e.g., via the opening 156) and the suction port 152 (e.g., via the first opening and the first lumen 162), and configured to engage the shelf or shoulder 170 when the suction valve 145 (e.g., and the valve stem 160) is in the second position to block flow or provide a fluid-tight seal between atmosphere and the suction port 152. In some examples, a second seal 179b of the suction valve 145 may be engaged with the housing 148 and the body 150 of the valve well 158 and remain stationary relative to the body 150 as the valve stem 160 translates relative to the body 150. In some examples, the second seal 179b may extend around a circumference of a distal opening 155 of the insert portion 151 of the housing 148. In one example, as depicted in FIG. 4, the second seal 179b may be annular, engaged with and/or coupled to the insert portion 151 of the housing 148, engaged with and/or coupled to the body 150 of the valve well 158, configured to engage the second portion 160b valve stem 160 when the suction valve 145 is in the first position to block a flow or provide a fluid-tight seal between the working channel port 154 (e.g., via the distal opening 155, the second opening 169 and the second lumen 164) and the suction port 152 (e.g., via side or radial opening 153, the first opening 167 and the first lumen 162), and configured to be spaced proximal of the second portion 160b of the valve stem 160 when the suction valve 145 is in the second position to allow flow between working channel port 154 and the suction port 152. In some examples, the first seal 179a may be configured to remain proximal of the first opening 167 when the suction valve 145 (e.g., and the valve stem 160) is in the first position and the second position and the second seal 179b may be configured to be distal of the first opening 167 when the suction valve 145 is in the first position and the second position. In some examples, the first seal 179a may be configured to be proximal of the second seal 179b when the valve stem 160 and the suction valve 145 are in the first position and/or the second position. Other suitable configurations of the first seal 179a and the second seal 179b are contemplated.

FIGS. 5 and 6 depict schematic side views of illustrative configurations of the valve stem 160 and the interface member 146. FIG. 5 depicts a schematic side view of the valve stem 160 and the interface member 146 at a first rotational position. FIG. 6 depicts a schematic side view of the valve stem and the interface member 146 at a second rotational position rotated ninety degrees from the first rotational position.

As depicted in FIGS. 5 and 6, the valve stem 160 may be formed from a solid component without any lumens or openings. The first portion 160a, the second portion 160b, and the third portion 160c may have any suitable dimensions configured to facilitate controlling flow paths through the suction valve 145. In some examples, the first portion 160a and the third portion 160c of the valve stem 160 may be configured to be inserted through an opening or lumen defined by the insert portion 151 of the housing 148. In some examples, the second portion 160b of the valve stem 160 may be configured to engage the second seal 179b when the valve stem 160 and/or the suction valve 145 is in the first position. In some examples, the first portion 160a may define a recess 171 configured to receive the first seal 179a. In the first rotational position depicted in FIG. 5, the third portion 160c of the valve stem 160 may have a width that may be less than a width of the second portion 160b of the valve stem 160. In some examples, the width of the third portion 160c of the valve stem 160 may be substantially equal to a width of the first portion 160a in the first rotational position, but other suitable configurations are contemplated. In the second rotational position, the third portion 160c of the of the valve stem 160 may have a width that is less than a width of the first portion 160a and the second portion 160b, as depicted for example in FIG. 5. In some examples, the width of the third portion 160c in the first position of the valve stem 160 may be a first width and the width of the third portion 160c in the second position of the valve stem 160 may be a second width that is less than the first width. Other suitable configurations of the third portion 160c and/or other portions of the valve stem 160 are contemplated.

The first portion 160a of the valve stem 160 may have a first cross-sectional area, the second portion 160b of the valve stem 160 may have a second cross-sectional area configured to create a seal engagement with the second seal 179b when the valve stem 160 is in the first position, and the third portion 160c of the valve stem 160 may have a third cross-sectional area configured to extend through the second seal 179b and allow fluid to flow between the valve stem 160 and the second seal 179b when the valve stem 160 is in the second position. In some examples, the third cross-sectional area may be less than the first cross-sectional area and/or the second cross-sectional area. In some examples, the third cross-sectional area may be constant along a length between the first portion 160a and the second portion 160b of the valve stem 160.

The valve stem 160 and/or the interface member 146 may be formed from any suitable materials. In some examples, the valve stem 160 may be formed from a first material and the interface member 146 may be formed from a second material, where the second material may be the same as or different than the first material. Example suitable materials for forming the valve stem 160 and/or the interface member 146 may include, but are not limited to, one or more of metals, polymers, acrylonitrile butadiene styrene (ABS), polycarbonate, and/or other suitable materials.

The seals 179 may be formed from any suitable materials. Example suitable materials for forming the seals 179 include, but are not limited to, one or more of a polymer, thermoplastic elastomers (TPE), thermoplastic polyurethane (TPU), liquid silicone rubber (LSR), and/or other suitable materials.

The material of the seals 179 may have any suitable durometer. In one example, the material of the seals 179 may have a durometer in a range of about 20-80 shore A, about 30-60 shore A, and/or other suitable values within one or more other suitable ranges of durometer, but could be softer or firmer depending on the geometry used for the seals and the amount of interference desired with other components of the suction valve 145. In one example, the one or more of the seals 179 may be formed from silicone with a durometer in a range of 40-50 shore A, but other suitable configurations of the seals 179 are contemplated.

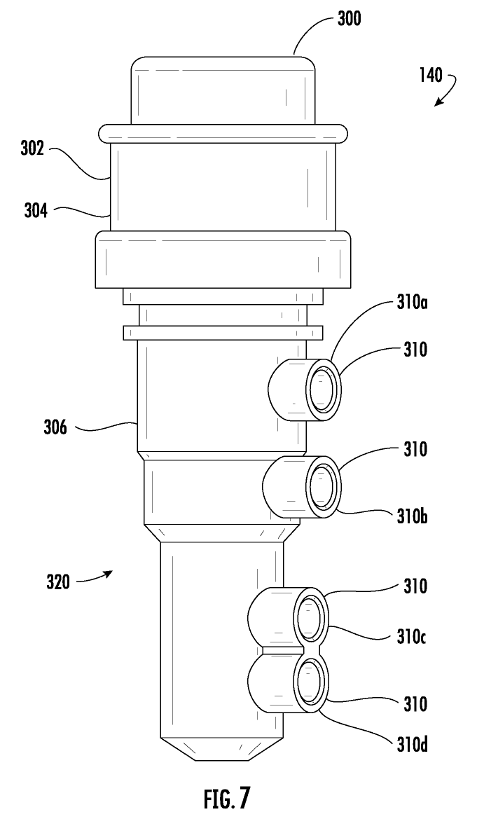

FIG. 7 depicts a schematic side view of an illustrative gas/water valve 140 configured for use in the endoscope 100. In some examples, the gas/water valve 140 may be used in a single-use configuration of the endoscope 100. In some examples, the gas/water valve 140 may include a valve stem (not shown in FIG. 7) configured to be maintained in or unremovable from a valve well 320 for the gas/water valve 140 of the endoscope 100. The gas/water valve 140 is unlikely to clog or require cleaning during use and as such, there may be no need to remove the gas/water valve 140 from the valve well 320 for cleaning or other purposes during us such that the valve stem of the gas/water valve 140 may be maintained within the valve well 320 during use and after use.

The gas/water valve 140 depicted in FIG. 7 may includes an interface member 300 (e.g., a cap, button, etc.), a housing 302 that may include a collar 304, and body 306 (e.g., a valve body) of the valve well 320, where the body 306 may define one or more ports 310. Although certain components of the gas/water valve 140 are depicted and described, other suitable components of the gas/water valve 140 are contemplated and/or certain depicted and described components of the gas/water valve 140 may be combined to form a single component and/or separated to form multiple components.

The body 306 may define or include any suitable number of ports 310. In some example and as depicted in FIG. 7, the body 306 of the valve well 320 may define a first port 310a, a second port 310b, a third port 310c, and a fourth port 310d, among other suitable ports 310. In one example, the first port 310amay be a gas outlet port configured to provide gas (e.g., air, etc.) to a working channel of the endoscope 100, the second port 310b may be a gas inlet port configured to receive gas from a gas source, the third port 310c may be a liquid outlet port configured to provide liquid to the working channel of the endoscope 100, and the fourth port 310d may be a liquid inlet port configured to receive liquid from a liquid source. Although the ports 310 are depicted on being on a single size of the body 306 and in a particular order, the ports 310 may be arranged at other suitable locations along the valve body 306.

In some cases, the housing 302 and the body 306 may form, be components of, and/or define the valve well 320. Alternatively or additionally, the housing 302 may be coupled with the valve well 320. Although the housing 302 and the body 306 are depicted as separate components, the housing 302 and the body 306 may be formed from a single component, as desired. Other suitable configurations of the housing 302, the body 306, and the valve well 320 are contemplated.

In operation, the gas/water valve 140 depicted in FIG. 7 (and a valve stem thereof) may have a first position or configuration (e.g., a relaxed or closed position) and one or more other positions or configuration (e.g., an actuated positions). Example gas/water valves and/or positions or configurations of the gas/water valves are described in U.S. Provisional Patent Application No. 63,661,217, filed on Jun. 18, 2024, and titled SEAL FOR AN ENDOSCOPE, which is hereby incorporated by reference in its entirety for any and all purposes.

FIG. 8 depicts a schematic bottom perspective view of the gas/water valve 140 depicted in FIG. 7. As depicted for example in FIG. 8, a distal end of the valve well 320 may have be closed and defined by the body 306.

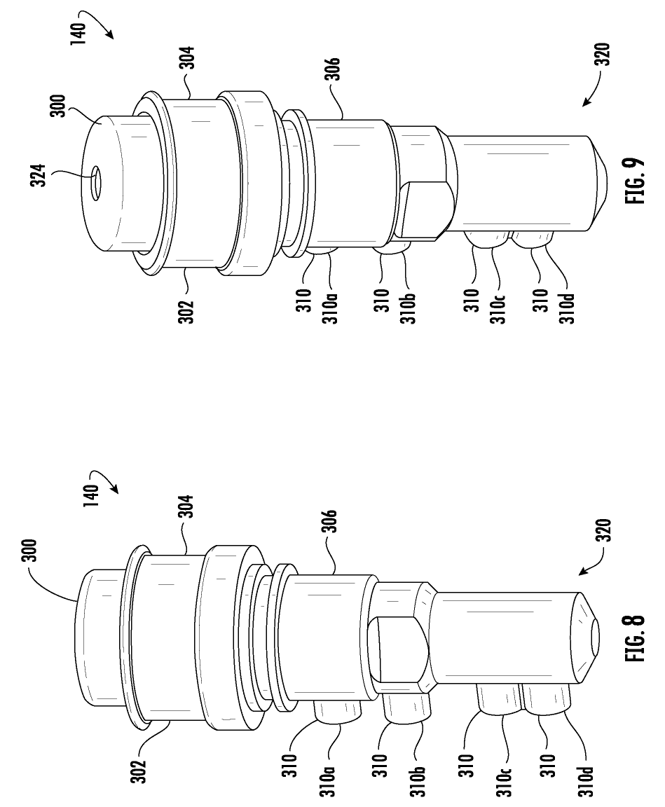

FIG. 9 depicts a schematic top perspective view of the gas/water valve 140 depicted in FIG. 7. As depicted in FIG. 9, the interface member 300 may include or define an opening 324 (e.g., a proximal opening, such as a gas escape hole and/or other suitable opening). In some examples, the opening 324 may be in fluid communication with a lumen extending through the valve stem of the gas/water valve 140.

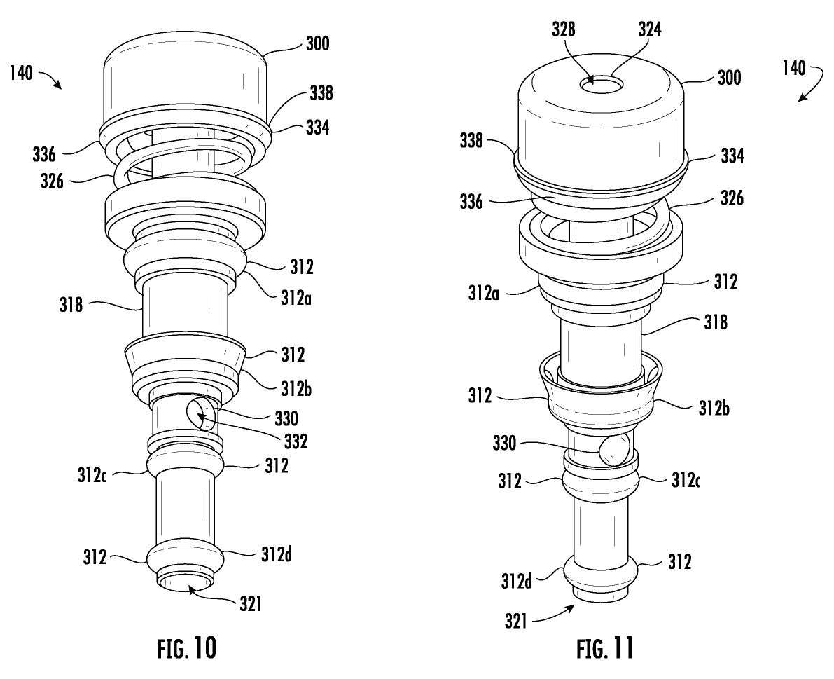

FIGS. 10 and 11 depict schematic bottom and top perspective views of components of the gas/water valve 140, without the body 306 of the valve well 320. As depicted in FIGS. 10 and 11, the gas/water valve 140 may include a valve stem 318 coupled with and/or extending from the interface member 300 and a biasing member 326 may extend between the interface member 300 and one or more components of the body 306 and/or configured to remain stationary relative to the body 306. The valve stem 318 and the gas/water valve 145 may be actuated between a first position and one or more second positions by applying a force on the interface member 300 to counter a biasing force of the biasing member 326 and removing the applied force to allow the biasing member 156 to bias and adjust the valve stem 318 to the first position.

In some examples, the valve well 320 (not depicted in FIGS. 10 and 11) may be configured to facilitate translation (e.g., longitudinal translation and/or rotational translation) of the valve stem 318 in the body 306 and/or the housing 302 in response to forces acting on the valve stem 318 via the interface member 300 to adjust a flow through the gas/water valve 140. In some examples, the valve well 320 may be in fluid communication with and/or define the one or more ports 310, where translation of the valve stem 318 within the valve well 320 may adjust flow paths through the gas/water valve 140.

The interface member 300 may be configured to couple with the body 306. In some examples, the interface member 300 may be configured to be inserted into the valve body 306 with and/or through the collar 304 and couple with the body 306 such that the interface member 300 and/or the valve stem 318 are not removable from the valve body 306 without damaging one or more components (e.g., the interface member 300, the collar 304, the body 306, the valve stem 318, etc.) of the gas/water valve 140. In some examples, once the interface member 300 has been fully inserted into the body 306 (e.g., through a proximal opening), the body 306 may block removal of the interface member 300 from the body 306, while allowing the interface member 300 to be actuated and/or adjusted between the first position and the one or more second position.

As depicted in FIGS. 10 and 11, the interface member 300 may include one or more first engagement features 334 and the body 306 may include one or more second engagement features (not depicted), where the first engagement features 334 may be configured to engage the second features when the valve stem 318 is in the first position to block removal of the interface member 300 from the body 306.

The first engagement features 334 and the second engagement features may have any suitable configurations configured to facilitate insertion of the interface member 300 in the body 306, actuation of the interface member 300 in the body 306, engage one another, and prevent removal of the interface member 300 and/or the valve stem 318 from the body 306. In some examples, the first engagement features 334 may be configured to extend around an outer circumference (e.g., a entirety of or at least part of the outer circumference) of the interface member 300 and the second engagement features may be configured to extend around an inner circumference (e.g., a entirety of or at least part of the inner circumference) of the body 306. The first engagement features 334 and/or the second engagement features may extend in a complete circle or may include spaces between adjacent portions of the engagement features. In one example configuration of the first engagement feature 334, the first engagement feature 334 may have a distal facing, beveled outer surface 336 that tapers inward in a distal direction. In some examples, the first engagement feature 334 may have a proximal shoulder 338 configured to engage the second engagement feature of the body 306 to prevent removal of the interface member 300 from the body 306.

The valve stem 318 may couple to the interface member 300 in any suitable manner. In some examples, a proximal portion of the valve stem 318 may be coupled to the interface member 300. For example, when the valve stem 318 is in communication with the interface member 300, the valve stem 318 may be releasably coupled with the interface member 300, monolithically formed with the interface member 300 with the same material(s) forming the interface member 300 (e.g., formed with a single material), fixedly coupled with the interface member 300, and/or may have one or more other suitable configurations with respect to the interface member 300 such that actuation of the interface member 300 may cause actuation or movement of the valve stem 318. Example suitable coupling mechanisms for coupling the valve stem 318 with the interface member 300 include, but are not limited to, adhesives, a threaded connection, a luer lock connection, a snap connection, a ball-detent connector, a friction fit, a forming procedure, a molding technique, and/or additional or alternative coupling mechanisms. In one example, the valve stem 318 may be monolithically formed with the interface member 300 using a single material. Other configurations of the valve stem 318 relative to the interface member 300 are contemplated.

The valve stem 318 may have any suitable configuration configured to adjust positions within the valve well 320, adjust flow paths to and/or from the ports 310, and couple with the interface member 300. For example, the valve stem 318 may be solid, may have or define one or more openings therethrough and/or therein, may have or define one or more lumens, may have or define one or more channels, and/or may have one or more suitable configurations. In some examples, the valve stem 318 may include one or more openings and one or more lumens in communication with the openings. For example, the valve stem 318 may include one or more first openings 328 at a proximal end of the valve stem 318 and aligned with and/or proximate the opening 324 of the interface member 300, one or more second openings 330 (e.g., radial openings), and one or more lumens 332 extending between and/or in fluid communication with the first opening(s) 328 and the second opening(s) 330. In some examples, the lumen may be co-axial with a central axis of the valve stem 318, but other suitable configurations are contemplated.

The valve stem 318 may couple with the interface member 300. In some examples, a portion (e.g., a proximal portion) of the valve stem 318 extending proximally of the one or more second openings 330 may be coupled to the interface member 300 via one or more suitable coupling mechanisms. Example suitable coupling mechanisms include, but are not limited to, adhesives, a threaded connection, a luer lock connection, a snap connection, a ball-detent connector, a friction fit, and/or additional or alternative coupling mechanisms.

The valve stem 318 may be formed in any suitable manner. In some examples, though not required, the valve stem 318 may be formed using a molding process, an injection molding process, a casting process, a finishing process, sanding, and/or by or with one or more additional or alternative manufacturing techniques. In one example, the valve stem 318 may be formed using an injection molding process.

The gas/water valve 140 may include any suitable number of seals along the valve stem 318. In some examples, four seals 312 may extend circumferentially around (e.g., extend partially or entirely circumferentially around) the valve stem 318 (e.g., around an elongate body of the valve stem 318) at different axial locations. In one example, a first seal 312a may be located between the first opening 328 and the second opening 330 at a location configured to be proximal of the first port 310a(e.g., a gas outlet) to obstruct flow in the gas/water valve 140 proximal of the location of the first port 310a, a second seal 312b may be located between the first seal 312aand the second opening 330 and may be configured to not impede gas flow when the valve stem 318 is in a first configuration (e.g., a relaxed, unactuated configuration), a third seal 312c may be located between the second opening 330 and the distal end 321 (e.g., a closed distal end) of the valve stem 318, and a fourth seal 312d may be located between the third seal 312c and the distal end 321 of the valve stem 318, but other suitable configurations are contemplated. Alternatively or additionally, the seals 312 of the gas/water valve 140 may be located along the valve body 306 such that the valve stem 318 may move relative to the seals.

The seals 312 may have any suitable configuration. In some examples, each of the seals 312 may include one or more wipers or flanges configured to engage an inner surface of the valve body 306, as depicted for example in FIGS. 8 and 9. In some examples, the features of two or more seals 312 may be combined into a single seal 312.

The components of the gas/water valve 140 may be formed from any suitable materials. In some examples, the valve stem 318 may be formed from a first material and the seals 312 of or coupled with the valve stem 318 may be formed from a second material, where the second material may be the same as or different than the first material. The valve stem 318 may be formed from a hard or rigid polymer and all of, part of one or more of, or one or more of the seals 312 may be formed from a flexible polymer, but other suitable configurations are contemplated. In some examples, the valve stem 318 may be formed from polymer, acrylonitrile butadiene styrene (ABS), polycarbonate, and/or other suitable material. Alternatively, the valve stem 318 may be formed of steel or aluminum. The seals 312 may be formed from one or more of a polymer, thermoplastic elastomers (TPE), thermoplastic polyurethane (TPU), liquid silicone rubber (LSR), and/or other suitable materials.

The material of the seals 312 may have any suitable durometer. In one example, the material of the seals 312 when positioned at the valve stem 318 may have a durometer in a range of about 20-80 shore A, about 30-60 shore A, and/or other suitable values within one or more other suitable ranges of durometer, but could be softer or firmer depending on the geometry used for the seals and the amount of interference desired with an inner surface of the body 306. In one example, the seals 312 may be formed from silicone with a durometer in a range of 40-50 shore A, but this is not required.

Additionally or alternatively to as discussed herein, the components of the suction valve 145 and/or the gas/water valve 140 may be formed in any suitable manner. For example, all or at least two or more of the components or portions of the suction valve 145 and/or the gas/water valve 140 may be formed monolithically of a single material, one or more components or portions of the suction valve 145 and/or the gas/water valve 140 may be formed separate from forming one or more other components or portions of the suction valve 145 and/or the gas/water valve 140, one or more components or portions of the suction valve 145 and/or the gas/water valve 140 may be embedded in one or more other components or portions of the suction valve 145 and/or the gas/water valve 140, and/or one or more components or portions of the suction valve 145 and/or the gas/water valve 140 may be formed in one or more other suitable manners. Example suitable coupling mechanisms include, but are not limited to, adhesive, a threaded connection, a luer lock connection, a snap connection, a ball-detent connector, a friction fit, and/or additional or alternative coupling mechanisms.

Additionally or alternatively to as discussed herein, any suitable techniques may be utilized to form the components or portions of the components herein. In some examples, though not required, the components or portions of the components herein may be formed using one or more of a molding process, an injection molding process, an over molding process, a casting process, a finishing process, sanding, and/or by or with one or more additional or alternative manufacturing techniques. In one example, the components or portions of the components may be formed using an injection molding process.

Additionally or alternatively to as discussed herein, the suction valve 145 and/or the gas/water valve 140 may include any suitable number of seals including those described herein. The seals may have any suitable configuration.

Additionally or alternatively to as discussed herein, the components of the suction valve 145 and/or the gas/water valve 140 may be formed from any suitable materials (e.g., the same or different materials). For example, the components herein may be formed from material including, but not limited to, metals, polymer, plastic, bioplastic, recyclable materials, acrylonitrile butadiene styrene (ABS), polycarbonate, thermoplastic elastomers (TPE), thermoplastic polyurethane (TPU), liquid silicone rubber (LSR), steel, aluminum, and/or other suitable material. In some examples, the seals herein may be formed from one or more of a polymer, thermoplastic elastomers (TPE), thermoplastic polyurethane (TPU), liquid silicone rubber (LSR), and/or other suitable materials. The material of the seals herein may have any suitable durometer.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The invention's scope is, of course, defined in the language in which the appended claims are expressed.

What is claimed is:

1. A valve assembly for use in a valve well of a medical device, comprising:
a housing;
an interface member;
a valve stem configured to adjust between a first position and a second position in response to actuation of the interface member relative to the housing;
a first seal coupled with the housing; and
a second seal coupled with the valve stem,
wherein the valve stem is configured to engage the first seal when the valve stem is in the first position and the second seal is configured to engage the housing when the valve stem is in the second position.

2. The valve assembly of claim 1, wherein the second seal is configured to be proximal of the first seal when the valve stem is in the first position and when the valve stem is in the second position.

3. The valve assembly of claim 1, wherein:
the housing comprises a radial opening,
the radial opening is distal of a proximal opening through the housing and is in fluid communication with the proximal opening through the housing when the valve stem is in the first position, and
the radial opening is proximal of a distal opening through the housing and is in fluid communication with the distal opening through the housing when the valve stem is in the second position.

4. The valve assembly of claim 3, wherein the first seal extends around a circumference of the distal opening through the housing.

5. The valve assembly of claim 3, wherein the interface member is located in the proximal opening.

6. The valve assembly of claim 1, wherein:
the valve stem has a distal portion, a proximal portion, and an intermediate portion between the proximal portion and the distal portion, and
the distal portion has a first cross-sectional area configured to create a seal engagement with the first seal when the valve stem is in the first position and the intermediate portion has a second cross-sectional area configured to extend through the first seal and allow fluid to flow between the valve stem and the first seal when the valve stem is in the second position.

7. The valve assembly of claim 6, wherein the proximal portion has a third cross-sectional area and the second cross-sectional area of the intermediate portion is less than the first cross-sectional area and the third cross-sectional area.

8. The valve assembly of claim 7, wherein the intermediate portion has a constant cross-sectional area between the distal portion and the proximal portion.

9. The valve assembly of claim 1, wherein the housing comprises a first portion configured to extend into the valve well and a second portion configured to receive the interface member.

10. The valve assembly of claim 9, wherein the first portion has a first inner diameter and the second portion has a second inner diameter that is greater than the first inner diameter.

11. The valve assembly of claim 1, further comprising:
a biasing member extending between the housing and the interface member,
wherein the biasing member is configured to bias the interface member to a first configuration with the valve stem in the first position.

12. A valve assembly for a medical device, comprising:
a valve body having one or more fluid inlet ports and one or more fluid outlet ports;
an interface member extending into the valve body;
a valve stem connected to the interface member and configured to translate within the valve body between a first position and a second position in response to actuation of the interface member, the valve stem comprising an elongate body and a central lumen extending through the elongate body between a first opening in the elongate body and a second opening in the elongate body; and
one or more seals extending around the valve stem and configured to block fluid flow between an outer surface of the valve stem and an inner surface of the valve body,
wherein the interface member is configured to be inserted into the valve body and the valve body is configured to block removal of the interface member from the valve body.

13. The valve assembly of claim 12, wherein the valve body comprises a proximal opening through which the interface member is inserted.

14. The valve assembly of claim 12, wherein the valve body comprises a first engagement feature and the interface member comprises a second engagement feature configured to engage the first engagement feature when the valve stem is in the first position to block removal of the interface member from the valve body.

15. The valve assembly of claim 14, wherein the second engagement feature comprises a beveled outer surface that tapers inward in a distal direction and a proximal shoulder configured to engage the first engagement feature.

16. The valve assembly of claim 14, wherein first engagement feature extends around an inner circumference of the valve body.

17. The valve assembly of claim 12, wherein the valve body is configured to receive the interface member and receive the valve stem.

18. A single-use medical device, comprising:
a handle;
an elongate shaft extending from the handle and configured to be inserted into a body cavity of a patient;
a suction valve configured to be removably positioned in a first valve body at the handle; and
a gas/water valve configured to be positioned in a second valve body at the handle, wherein the second valve body is configured to block removal of the gas/water valve from the second valve body.

19. The single-use medical device of claim 18, wherein the suction valve comprises:
a housing inserted into the first valve body;
a valve stem configured to adjust between a first position and a second position relative to the first valve body;
a first seal coupled with the housing; and
a second seal coupled with the valve stem,
wherein the valve stem is configured to engage the first seal when the valve stem is in the first position and the second seal is configured to engage the housing when the valve stem is in the second position.

20. The single-use medical device of claim 18, wherein the gas/water valve comprises:
an interface member extending into the second valve body; and
a valve stem connected to the interface member and configured to translate within the second valve body between a first position and a second position in response to actuation of the interface member,
wherein the second valve body comprises a first engagement feature and the interface member comprises a second engagement feature configured to engage the first engagement feature when the valve stem is in the first position to block removal of the gas/water valve from the second valve body.
